(19) United States
(12) Patent Application Publication
(10) Pub. No.: US 2008/0318876 A1
(43) Pub. Date: Dec. 25, 2008

(54) Use of Ginsenosides in the Treatment of Aids

(76) Inventors: Zelin Li, Beijing (CN); Yue Zeng, Beijing (CN); Yi Zeng, Beijing (CN); Xin Zeng, Beijing (CN)

Correspondence Address: CONLEY ROSE, P.C.;David A. Rose, P. O. BOX 3267, HOUSTON, TX, 77253-3267, US

(21) Appl. No.: 11/574,855

(22) PCT Filed: Sep. 8, 2005
(86) PCT No.: PCT/CN2005/001429

§ 371 Date: Aug. 7, 2008

(30) Foreign Application Priority Data
Sep. 8, 2004 (CN) 200410074307.2

(51) Int. Cl. A61K 31/704; A61P 31/12

(52) U.S. Cl. 514/26

(57) ABSTRACT

The present invention relates to the use of compounds of formula (I) in the treatment of AIDs,

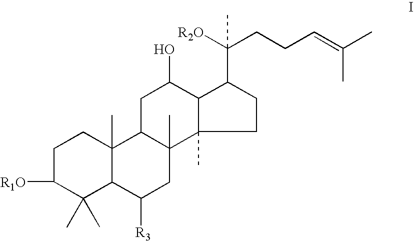

wherein, R₁ is -Glc²-Glc, R₂ is selected from the group of -Glc⁶-Glc, -Glc⁶-Ara(p), -Glc⁶-Xyl and -Glc⁶-Ara (f); and R₃ is H, and also relates to the composition comprising the above compounds and the use of the herbal extracts in the treatment of AIDS.

### FIELD OF THE INVENTION

The invention relates to a drug for treating acquired immune deficiency syndrome, AIDs, specifically, the invention relates to the use of Ginsenosides extracted from Ginseng for treating AIDs. The invention also relates to the use of Ginsenosides-containing composition for the treating AIDs. Furthermore, the invention relates to the use of Ginsenosides-containing root, stem and leaf extracts of plants selected from Panax ginseng C. A. Mey, Radix panacis quinquefolii, gymostemma pentaphyllum (thumb) makino, Panax notoginseng (burk) F. H. Chen, Panax japonicus C. A. Meyer and Panax japonicus var: major (burk.) Wu et Feng in the treatment of AIDs.

### BACKGROUND OF THE INVENTION

With sweet and slightly bitter tastes as well as a mild action, the ginseng roots are capable of adjusting the energy flows, nurturing the blood, quieting the spirits, sharpening the wisdom, generating the saliva, relieving the cough, replenishing the nutrients and strengthening the body. Known as “the King of Herbs”, it has been the preferable choice of body-regulating drug ever since the ancient times in China. It is reported that Ginseng has the following efficiency:
1. Ginseng has a sedative effect on central nervous system, and antagonistic action to a number of analeptic, and reduces inhibitory effect of central depressive drugs. Rb series compounds of Ginsenosides have sedative effect on central nervous system, whereas Rg series compounds have weak excitable effect, but shows inhibitory effect under over-dose. Ginseng can not only improve the excitable process of central nervous system, but also strengthen the inhibitory process, together with centralizing inhibition and differentiating completely.
2. Ginseng has anti-fatigue effect. Panaxadiol, panaxtrol and all kinds of Ginsenosides have effect of anti-fatigue, wherein the activity of panaxtrol is more than 2 times of that of panaxadiol. It is believed that ginseng pasty extract liquid can promote economic use of glycogen and high energy phosphate compound, can enhance the metabolism of lactic acid and pyruvic acid, and provide energy timely by oxidation for the muscular movement.
3. Ginseng increases blood pressure with low dose and lowers blood pressure with high dose for anesthetic animals. But a therapeutic dose has no effect on patient's blood pressure; Ginsenosides have slightly and shortly effect on lowering blood pressure. Ginseng water extract has the same effect as cardiac glycosides, strengthening the contraction frequency and slowing heart rate.
4. Ginseng enhances nonspecific resistance of human body to many deleterious factors. It can, for example, strengthen the resistance of human body to invasion from physical (cool, high temperature, over exercise, high pressure or low pressure), chemical (all kinds of toxicants, narcotics), biological (foreign serum, microorganism, transplanting tumor) factors,
5. Ginseng has blood glucose reduction effect in normal rabbits, as well as in rats and dogs induced to high blood glucose with alloxan tetrahydrate, adrenalin, but nevertheless cannot be used as the substitute of insulin.
6. Ginseng has the effect like gonadotropin, wherein ginsenosides A, C, F all have the activity of gonadotropin with similar intensity.
7. Panax Ginseng PE has no activity of hemolysis, but weak anti-hemolysis effect that is induced by Ginsenosides or lecithin. It is reported recently, the elements of Rh, Rg and Rf of ginsenosides (that is, saponins with panaxtrol being the genin) have the activity of hemolysis, whereas the elements of Rc and Rb (that is, saponins with panaxdiol being the genin) have the activity of anti-hemolysis.
8. Injection prepared by ginseng extract can increase the content of erythropoietin in bone marrow of the rabbit. No matter being orally administrated or externally applied, Ginseng extract can improve the biologically synthesis of DNA, protein, lipid in the bone marrow cell, at least parts of the active elements of the Ginseng extract are Ginsenosides (especially Rb2, Rg1, etc). After being administrated intra-gastricly, Ginseng extract exhibits the activity of antidiuresis.
9. Ginseng has favorable effect on the function of pituitary adrenal system, showing that it can not only enhance the resistibility of animals to disadvantage conditions (such as high temperature, low temperature, long time swimming, etc), but also alleviate the change of adrenal hypofunction induced by stress reaction.
10. Ginseng can also improve the synthesis of protein and nucleic acid. It has been reported recently that Ginseng extract can greatly improve the synthesis of nucleic acid and protein in liver, kidney, bone marrow, spermary cells of rats, as well as the synthesis of serum protein.

As demonstrated by many experiments, ginseng can enhance the physical and intellectual activities of animals and human and boost the body's non-specific resistance against a variety of noxious stimulations. Within therapeutic range, it has no interference with the normal physiological functions and no side effects. It is considered as a class of beneficial and harmless strengtheners and tonics for the whole body.

According to the discovery of the said pharmacological effects of ginseng, the researchers have conducted researches of the ginseng extracts. It is found that more than 10 kinds of Ginsenosides exist with major pharmacological actions, such as Rb1, Rb2, Rb3, Rc, Rd, Re, Rg, Rh1, Rh2, F2, pseudo-ginsenoside F1, RTs and American Ginsenoside L1. The main pharmacological studies of these Ginsenosides include the effects of anti-aging, immunity-boosting and blood lipid-lowering, and some changes of heart and blood vessels. But until now, there is still no report of employing ginseng, ginseng extracts or any Ginsenoside to treat AIDS. The causative agent of AIDS is Human Immunodeficiency Virus (HIV), which mainly attacks the human immune system, especially the CD4 lymphocyte. At last, the body's immune functions are destroyed, resulting in opportunistic infections and causing the patient's death.

Suramin was the first reported drug to fight against the HIV. In 1985, AZT was found to possess the anti-HIV activity in vitro. In 1986, the clinical trial was carried out. In 1987, AZT became the first drug approved by FDA for treating AIDS. But the main issues were its drug toxicities and resistance. Other drugs appeared in the subsequent years. Until now, more than twenty anti-HIV drugs have been approved for commercial uses in the US. According to their mechanisms of action, they fall mainly into 3 categories. Except for T20-blocking the entry of HIV into the cells approved at the end of Year of 2002, all the other drugs belong to the viral reverse transcriptase (RT) inhibitors, such as AZT, DDC, DDI and viral protease inhibitors. FDA had already approved five drugs of protease inhibitors, namely Saquinavir, Ritonavir, Indinavir sulfate and Nefinavir, etc. In 1995, the American scientists adopted the “Triple Combination” regimen of two RT inhibitors and one protease inhibitor. Known as HAART, such therapy is currently in common uses. This therapy has improved the treatment outcome and further prolonged the patient's life. It has been now used for 10 years, several patients still survive.

There are currently three drugs available for the cocktail of HAART in China. But there are only RT inhibiting drugs that have toxicity so serious that nearly 20% of the patients cannot tolerate. Therefore there exist the issues of drug toxicities and resistance in the clinical applications. As stated above, T20 is able to block the entry of virus into the cells. However, T20 cannot be taken orally since it is a peptide. It must be injected for application. And the price is quite expensive. Therefore it is imperative to develop anti-HIV drugs with low toxicities and ability of inhibiting drug-resistant HIV.

The traditional Chinese medicine is such a great treasure-house that it is quite worth to be explored and carried forward. Through more than ten years' researches, the inventors have found out that some herbal extracts, ingredients or mono-components have well-defined anti-HIV activities. The targets of their anti-HIV activities were studied. With such advantages as a cheaper price and lower toxicities over HAART, they can markedly boost the immune functions. And it is necessary to constantly update the therapeutic cocktail since HAART has the problem of drug resistance. So there are broad prospects of applying traditional Chinese medicine to treat AIDS in clinical settings.

### SUMMARY OF THE INVENTION

The present inventor has found out that dammarane type compounds (also is known as tetracyclic triterpene dammarane) among ginseng extracts can be used for treating AIDS with better efficiency.

Therefore, on one hand, the present invention relates to the use of compounds of formula I in the preparation of drugs for treating AIDS:

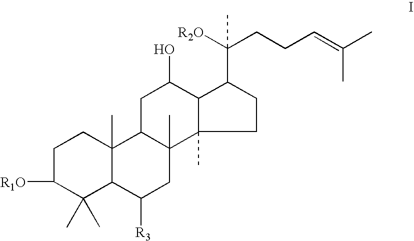

wherein, R₁ is -Glc²-Glc, R₂ is selected from the group of -Glc⁶-Glc, -Glc⁶-Ara(p), -Glc⁶-Xyl and -Glc⁶-Ara(f); and R₃ is H. Preferably, R₁ is -Glc²-Glc, R₂ is -Glc⁶-Xyl, and R₃ is H.

The prefer dose of present compounds is in amount of 0.03-0.50 mg/kg body weight.

Present compounds can be administered through oral way, intra-dermal way, injection, inhale or mucosal way.

Present compounds may be used in combination with other anti-HIV drugs in a synergistic way including such as AZT, DDC, DDI, Saquinavir Ritonavir, Indinavir sulfate and Nefinavir or combination thereof preferably AZT. Said compounds are also active against HIV virus that is resistant to some drugs repeatedly used.

On another aspect, present invention relates to the use of pharmaceutical composition comprising the compounds of formula I as active ingredient in the preparation of drugs for treating AIDs:

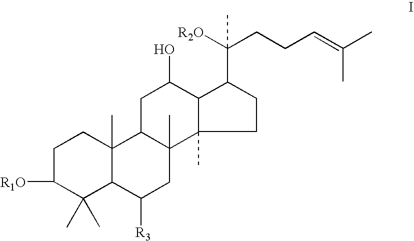

wherein, R₁ is -Glc²-Glc, R₂ is selected from the group of -Glc⁶-Glc, -Glc⁶-Ara(p), -Glc⁶-Xyl and Glc⁶-Ara(f); and R₃ is H.

Furthermore, present invention also relates to the use of root, stem or leaf extracts of Panax ginseng C. A. Mey, Radix panacis quinquefolii, Gymostemma pentaphyllum (thumb) makino, Panax notoginseng (burk) F. H. Chen, Panax japonicus C. A. Meyer and Panax japonicus var: major (burk) Wu et Feng and/or dry powder thereof in the preparation of drugs for treating AIDs, said extracts comprise the compound of formula I:

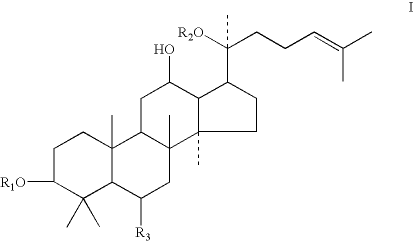

wherein R₁ is -Glc²-Glc, R₂ is selected from the group of -Glc⁶-Glc, -Glc⁶-Ara (p), -Glc⁶-Xyl and -Glc⁶-Ara(f); and R₃ is H.

The compounds of formula I disclosed in present invention and/or extracts comprising the compounds of formula I are all active to the virus resistant to AIDs. Said extracts may be obtained from the root, stem and/or leaf of the plants mentioned above, still further, dry powder made from these plants is also included in the present technical solution of the invention. The extracts made from the root, stem or leaf of Panax ginseng C. A. Mey, Radix panacis quinquefolii, Gymostemma pentaphyllum (thumb) makino, Panax notoginseng (burk) F H Chen, Panax japonicus C. A. Meyer and Panax japonicus var: major (burk) Wu et Feng can be used in combination with the dry powder of these plants.

Present invention also relates to a pharmaceutical composition comprising present compounds or extracts as active element, and ordinary pharmaceutically acceptable excipient or supplementary agents.

Present compounds may be prepared by well-known methods of this field. For this purpose, if necessary, present extracts or compounds may be combined with one or more solid or liquid excipients to prepare into suitable administration formulation.

Present extracts or compounds or pharmaceutical composition comprising thereof may be administrated in unit dosage form, and administrating method may be intestine or parenteral, such as oral, muscle, subcutaneous, nasal, bucccal mucous member, skin, peritoneum or rectum etc, preferably oral.

The administrating method of present extracts or compounds or pharmaceutical composition comprising thereof may be injection including mainly intramuscular injection, hypodermic injection and intradermal injection.

The dosing formulations may be liquids or solids. For example, the liquid formulations can be true solution types, colloid types, microgranular forms, emulsion forms and suspensive forms. Other formulations include tablets, capsules, drops, aerosols, pills, pellets, solutions, suspensions, emulsions, granules, suppositories and freeze-dry powder injections and the like.

Present extracts or compounds may be made into common preparations, sustained-release preparations, controlled-release preparations, targeted preparations and varieties of mircosomal drug delivery systems.

All carriers known in the art can be used so as to prepare the unit dosage formulations into the tablets, e.g. carriers as dilutes and absorbents including starch, dextran, calcium sulfate, lactose, mannitose, sucrose, sodium chloride, glucose, urea, calcium carbonate, white clay, microcrystal cellulose and aluminum silicate, etc; lubricant and adhesive, such as water, glycerol, polyethylene glycol, ethanol, propanol, starch slurry, dextran, syrup, honey, glucose solution, Arabian glue, gelatin glue, carboxylmethylcellulose sodium, sdshellac, methylcellulose, potassium phosphate and PVP, etc; disintegrant, such as dry starch, alginate, agrose powder, algin starch, sodium bicarbonate, citrate, calcium carbonate, polyoxyethylene sorbitan alphate, sodium dodecylsulfonate, methylcellulose and ethylcellulose, etc; disintegration inhibitor, such as sucrose, glycol tristearate, cocoa butter and hydrogenated oil, etc; absorption promoter, such as quaternary ammonium and sodium dodecylsulfate, etc; lubricant, such as talcum powder, silicon oxide, corn starch, stearate, borate, liquid paraffin and polyethylene glycol, etc. Other carriers, such as polyacrylic acid resins and liposome; water-soluble carriers, such as PEG4000, PEG6000 and PVP, etc can also be used. Also the tablets may be further prepared into coated pills, such as sugarcoated, thin membrane-coated, enteric coated, or double-layered and multi-layered tablets.

For example, it is feasible to adopt extensively all the known carriers in the art so as to prepare the unit dosage formulations into the pellets. Examples of carriers are dilutes and absorbents, such as glucose, lactose, starch, cocoa butter, hydrogenated plant oil, PVP, kaolin and talcum powder, etc; adhesive, such as Arabian glue, tragacanth gum, gelatin, ethanol, honey, liquid sugar and rice or flour paste, etc.; disintegrant, such as agrose powder, dry starch, alginate, sodium dodecylsulfonate, methylcellulose and ethylcellulose, etc.

For example, with the purpose of preparing the unit medicine into the capsules, the active ingredients of the invented medicinal composition can be mixed with various said carriers to obtain a mixture. And the mixture is placed into hard gelatin or soft capsules. It is also feasible to prepare the active ingredient of present compounds into microcapsule, into suspensions by suspending into aquose medium or into hard capsules or injection formulation.

For example, present extracts or compounds may be prepared to be injections in form, such as solutions, Suspensions, emulsions and freeze-dry powder injections. This kind of preparation can contain water or no water, may contain one and/or multiple pharmacologically acceptable carriers, dilutes, adhesives, lubricants, preservatives, surfactants or dispersers. For dilutes, water, ethanol, polyethylene glycol, 1,3-propylene glycol, ethoxylated prisorine, polyoxylated prisorine and polyoxyethylene sorbitan alphate are included. In addition, it is feasible to add appropriate amounts of sodium chloride, glucose or glycerol into the injections to prepare the isotonic injections. Furthermore the conventional solubilization boosters, buffering agents and pH adjusting agents may be added. These supplemental materials are commonly used in this field.

Besides, if necessary, the colorants, preservatives, spices, taste modifiers, sweeteners and other substances may be also included in the present pharmaceutical formulations.

For the purposes of achieving the medication goals and improving the therapeutic effects, present drugs or composition may be administered by any well-known dosing method.

The dosages of the present compounds or composition are determined by many factors, such as the severity of disease courses for the AIDS patients, sex, age, body weight, disposition, individual response, dosing routes, dosing frequency and treatment goals. As a result, the therapeutic dose range of this invention is large in change. Generally speaking, the practical doses of drug ingredients of this invention in the traditional medicine are well known among the professionals in this field. It is possible to make adequate adjustments to the actual drug quantity of the final preparation of present compounds or composition so as to achieve the effective therapeutic levels and accomplish the preventive or therapeutic goals of this invention. The proper daily dose range of the medicinal herbs composition is in amount of 0.03-0.50 mg/Kg body weight. The above dose may be administered in 2, 3 or 4 times per day. The administration is subject to the clinical experiences of physicians and influenced by the dosing plans through other therapeutic approaches.

It is also possible to prepare those compounds that are more active, fewer amounts found in present invention by semi-synthesis method to provide sufficient amount of said compounds for pharmaceutical use.

### BRIEF DESCRIPTION OF DRAWINGS

FIG. 1 is analysis figure showing the target of Rb3 on inhibiting HIV in vitro

FIG. 2 A, B are figures showing the binding of JHR, Rbs to protein gp41.

FIG. 3 is figure showing the binding of Rb3 to protein gp120.

FIG. 4 is figure showing no action of JHR on CD4 receptor, wherein A is for control, B is for administration.

FIG. 5 is figure showing the effects of the JHR upon the CXCR4 and CCR5 co-receptors.

FIG. 6 is analysis figure showing the target of Rb3 on inhibiting HIV in vitro.

### DETAILED DESCRIPTION OF THE INVENTION

Two aspects were considered by the inventors when carried out research on the anti-AIDs drugs, one was modern medicine which puts emphasis on the importance of anti-virus in the treating of AIDs, another was the effect of drugs on immune function. It is desired, in combination with the traditional medicinal theory, to provide drugs that have anti-virus activity and enhanced immune function, and so to achieve the aim of adjusting the energy flows, nurturing the blood, quieting the spirits, sharpening the wisdom, generating the saliva, relieving the cough, replenishing the nutrients and strengthening the body, and increasing CD4 cells. As a result, the inventors found the compounds of formula I

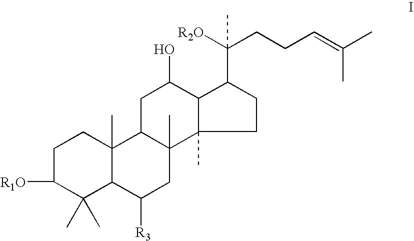

wherein, R₁ is -Glc²-Glc R₂ is selected from the group of -Glc⁶-Glc, -Glc⁶-Ara(p), -Glc⁶-Xyl and -Glc⁶-Ara(f); and R₃ is H. Preferably, R₁ is -Glc²-Glc, R₂ is -Glc⁶-Xyl, and R₃ is 1H.

Present compounds are used for treating AIDs.

Compounds of formula I are well known, and may be extracted or synthesized according to the method reported in prior art.

Specifically, the prefer compounds of present invention include Rb1, Rb2, Rb3, Rc and the like, wherein Rb2 and Rb3 are known isomers having the same structure.

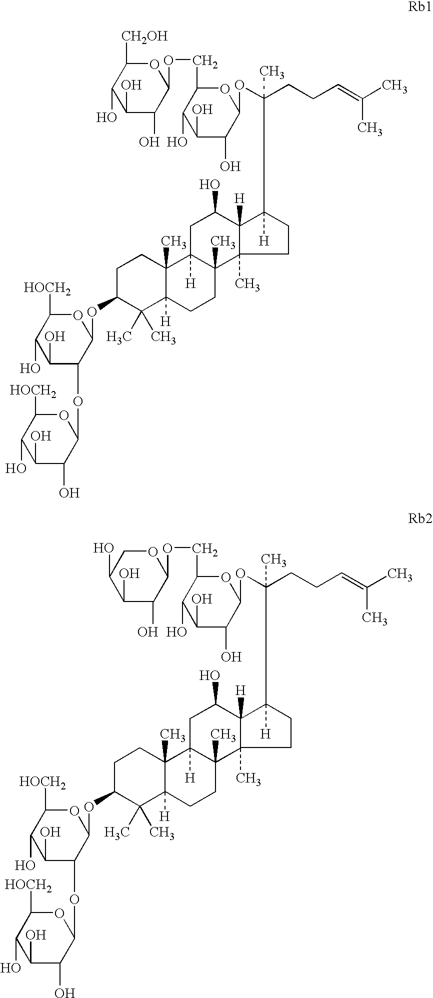

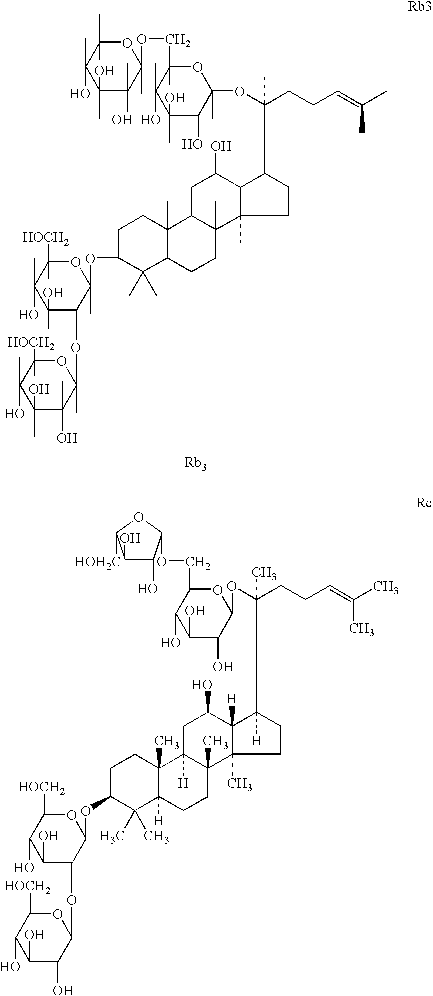

Said compounds of formula I were extracted first from ginseng. It is found however that many kinds of plants comprise this kind of compounds, said plants include but do not be limited to: Panax ginseng C. A. Mey, Radix panacis quinquefolii, Gymostemma pentaphyllum (thumb) makino, Panax notoginseng (burk.) F H Chen, Panax japonicus C. A. Meyer and Panax japonicus var: major (burk) Wu et Feng, as well as any other one that comprises the compounds of formula I. And the compounds of formula I are not only exit in the root of above plants, but also in the stem and leaf in a sufficient amount.

Present composition comprises more than one compounds of formula I:

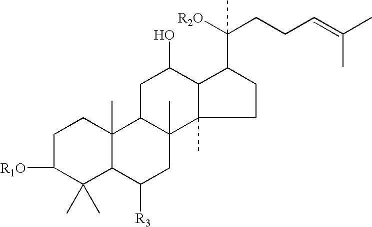

wherein, R₁ is -Glc²-Glc, R₂ is selected from the group of -Glc⁶-Glc, -Glc⁶-Ara(p), -Glc⁶-Xyl and -Glc⁶-Ara(f); and R₃ is 1.

The prefer ratio of the components in present composition are: Rb1:15-20 wt %, Rb2:15-20 wt %, Rb3:30-90 wt %, Rc:30-90 wt %.

Therefore, skilled person in the art will understand that since the compounds with formula I have the activity of anti-AIDs, any plant comprising the compound can also be used to treat AIDs, and as a result, herb composition, mixture or grindings that comprises said compound of formula I are all covered by present protection scope.

The following examples are to explain that present compounds have the activity of anti-AIDs virus, but not to limit its scope in any manner.

### EXAMPLE 1

### In Vitro Experiment

Three types of cells (such as MT4, Hela-CD₄, PBMC) were infected respectively with HIV-1 virus for observation of the inhibiting effects of ginseng composition (JHR) prepared in Example 1 upon the HIV-1 replication.

### (I) MT4 Cells

### Viral Strains: NL4

Method: A ginseng composition was prepared into a concentration of 1 mg/ml as drug solution and diluted into different concentrations for later uses during the experiment. Based on the analysis, the ginseng composition comprises 15 wt % Rb1, 33 wt % Rc, 17 wt % Rb2 and 35 wt % Rb3.

The experiment was carried out on a 96-well culture plate, 100 μl drug solution was added into each well and making each concentration's drug solution at least duplicate.

In the tube 5×10⁶ MT4 cells in were infected with HIV-1 (1×10⁴ TCID₅₀ in 1 ml medium), then cultured in the incubator with 5% CO₂ at 37° C. for 2 hrs, the HIV-1 infected cells were centrifuged, the supernatant was discarded and washed once with RPMI₁₆₄₀ to remove the free virus, 10 ml complete medium was added to the HIV-1 infected cells to make cells suspension (HIV-cell), 100 μl this cell suspension was added into each well which has contained drug solution, then the 96-well culture plate was cultured at 37° C. within a CO₂ incubator. On the third day, 100 μL supernatant was sucked out from each well replaced by 100 μL of medium with the same concentrations of drug as one in the wells. 100 μL of culture medium was added as a control group. On the sixth day, the supernatant was taken from each well. The Microelisa method and reagents were adopted to measure the amount of P₂₄ antigen. For each experiment, the virus control (VC), cell control and AZT positive drug control were used. Based upon the amount of P₂₄ antigen (P₂₄-Ag), the inhibition rate (IR) was calculated according to the following formula:

$$I R = \frac{V C well {P}_{24} \text{-} Ag - Drug group {P}_{24} \text{-} Ag}{V C well {P}_{24} \text{-} Ag} \times 100$$

Different IRs were obtained for drug solutions of different concentrations. After statistic processing, IC₅₀ was obtained.

To MT4 cells, IC₅₀ of ginseng composition JHR is 105.2 μg/ml

### (2) Hela-CD4 Cells

Virus: Single-life-cycle reporter HIV was acquired by transfection with HIV plasmids.

Method: Hela-CD₄-LTR-gal cell was inoculated into a 24-well plate at 0.4×10⁵/well and cultured for 24 hours and let the cells absorb and adhere to the wall. On the second day, the supernatants were sucked away from the wells and 100 μl drug (drug control) or drug (drug solutions with different concentrations) and HIV-1 or culture medium (Mock) were added. After 2 hours, 200 μl of identical drug solutions or culture medium was added into each well for culturing at 37° C. in a CO₂ incubator for 48 hours and detecting by the following method.

Fixation: The supernatants were sucked away from each well and adding the fixative solution (1 ml), and then by staining with K₄[Fe(CN)₆].3H₂O, K₃[Fe(CN)₆] and X-gel.

Counting: For the blue cell counts (BCC) within each well, the following formula is used to calculate the IR and then IC₅₀.

$$I R = \frac{Mock well B C C - Drug well B C C}{Mock well B C C} \times 100$$

TABLE 1
In vitro: inhibiting the replication of HIV-1
in the cell (HeLa-CD4 cell) (MAGI test)

| Dose | (mg/ml) | Inhibition rate (%) |
|---|---|---|
| 1. | 0.5020 | 100 |
| 2. | 0.1255 | 98.8 |
| 3. | 0.0313 | 26.2 |
| 4. | 0.0078 | 0 |

Result: To Hela-CD4 cell strain IC₅₀ of ginseng composition JHR is 44.3 μg/ml.

### (3) PBMC Cells

Virus: NL4-3
Method: The freshly harvested PBMC (human peripheral blood newly isolated lymphocytes) was collected, following by counting and centrifuging at 1200 rpm, discarding the supernatant and preparing the medium with 3×10⁶ cell/ml. The culture medium was pre-treated with IL2 (1 μl 1000×IL2 for every ml culture medium) at 37° C. overnight. For the experiment, 5×10⁶ was counted as one infection unit. The duplicate series of JHR at the same concentration of 0.4 mg/ml, virus control and cell control were set up. On a 24-well plate, each well contained 5×10⁶ cells in 0.5 ml drug solution or culture medium. NL4-3 virus (HIV-1) with the viral load of 4×10⁴ IU in each well was mixed and transferred into a 12-well plate. Then 1.5-ml of the identical drug solution or culture medium were added and incubated at 37° C. The supernatant was taken every 3-4 days in 100 μl Fetched for each well and stored at −80° C. RT was measured and the drug group was compared with virus control group to calculate the inhibition rate.

Result: Ginseng composition has outstanding inhibition activity to the HIV-1 of PBMC cell in vitro test shown in Table 2.

TABLE 2

| Days after administration | inhibition rate (%) |
|---|---|
| 3 days | 73.59 |
| 6 days | 100 |
| 9 days | 100 |
| 12 days | 100 |

### EXAMPLE 2

The same method as that in example 1 is applied, except that the drug added into the wells is Ginsenoside monomer, and the concentration thereof is listed in table 3.

### (I). Analysis and Comparison of Anti-AIDs Effect of Ginsenoside Monomer-Hela-CD4 Cell Strain

TABLE 3

| | Inhibition Rate | Inhibition Rate |
|---|---|---|
| Compound | (0.2 mg/ml) | (0.4 mg/ml) |
| Rb1 | 41.88% | |
| Rb2 | 42.93% | |
| Rb3 | 70.68% | 97.9% |
| Rc | 60.73 | |
| Rd | 9.42% | |

It can be seen from the above table, among five Ginsenoside extracts, Rb3 has the best inhibition rate of anti-AIDs.

### (II) Action Targets of Rb3

Object: It is to observe which phase(s) of viral life cycle are targeted by the said ginseng composition, including viral entry into cells, reverse transcriptase, integrase, transcription and proteases. The viral “single life cycle” model was employed to study the target of drug actions.

(1) MAGI method: The recombinant virus has the LTR of HIV. The reporter gene of β-galactosidase was expressed to form one kind of vital “single life cycle” model. As mentioned as above, this model employs K₄[Fe(CN)₆], K₃[Fe(CN)₆] and x-gel to stain the Hela-CD₄ cell. The blue cells under the microscope denote the presence of viral replication.

### (2) Luciferase Method

The recombinant and transfected VSVG virus and the cell line of H9 strain were adopted. The examination method was used to detect the activity of Luciferase by illumination. A heavier viral load denoted a higher enzymatic activity.

Experiment method: After viral infections, the cells were divided into different groups. They were also dosed respectively at 0, 6, 12, 18, 24 and 36 hours after infections. 48 hours after infection, the method of MAGI or Luciferase was adopted.

The above two testing methods were both viral models of “single life cycle”. Their major advantages showed that different infection times denoted different phases of viral replications. At 2-6 Hours, the virus was entering the cell. At 10-14 Hours, it was the phase of reverse transcription. After 20 Hours, it was the phase of recombination and transcription. As a result, dosing at different time points acted upon specific target points. This invention experiment had analyzed different target points of JHR and Rb3.

TABLE 4
Analysis on the viral life cycle stage(s) targeted
by drug of Rb3 disclosed in present invention

| Days after dosing | | | |
|---|---|---|---|
| (post-infection) | 2 hours | 10 hours | 29 hours |
| Inhibition rate (%) | 83.98 | 0 | 0 |

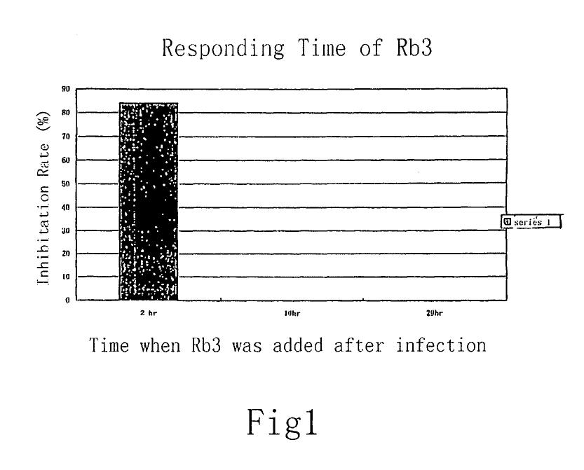

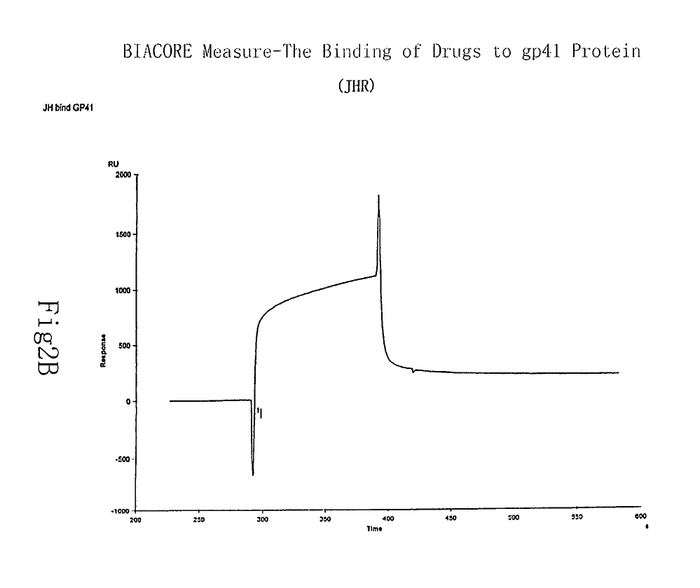

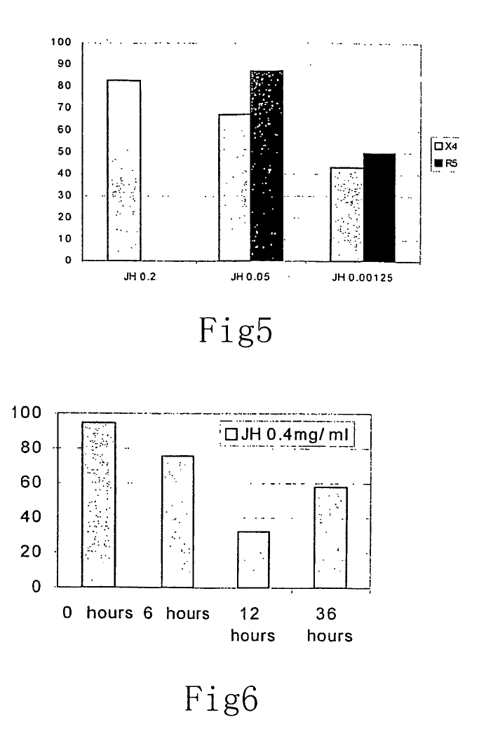

The result is shown in FIGS. 1 and 6, and it can be seen from the figures that the best inhibition effect was obtained when the drugs was administrated after 2 hours of the infection, with the target at that time is to block the viral to entry into the cells.

Macrophage-tropic lymphocytes were used to show the action on CCR5 co-receptor, and T lymphotropic lymphocytes are used to show the action on CXCR4 co-receptor.

(III) The use of BIACORE in Measuring the Binding of JHR, Rb3 to gp41, gp120 Protein
1. The Binding of Rb3 to gp41 Protein

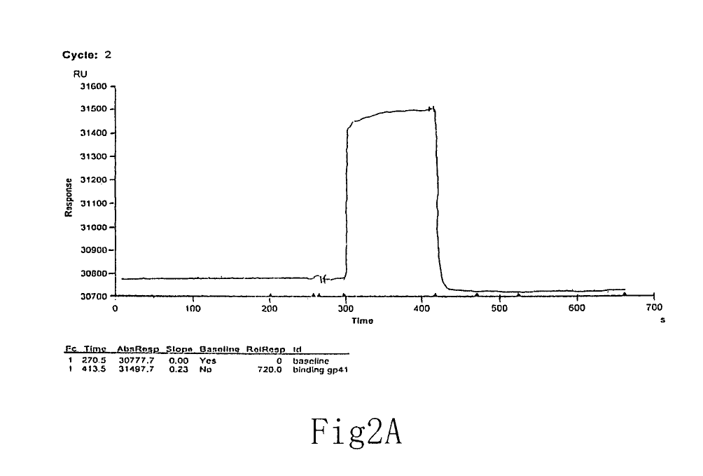

The genetically recombined gp41, gp120 were placed on the chip of BIACORE analyzer, respectively, after adding a certain concentration of Rb3 in sufficient amount, the instrument could detect whether or not there was a binding. The result is shown in FIG. 2

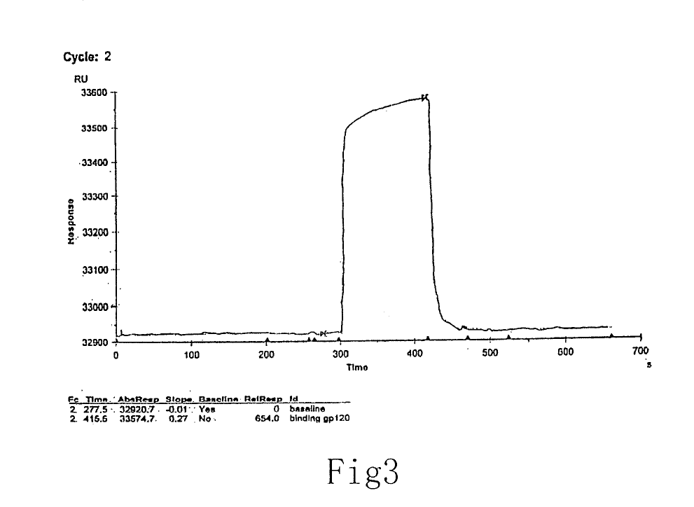

2. The Binding of Rb3 to gp120 Protein, the Result is Shown in FIG. 3.

From the result, it can be seen that both JHR and Rb3 can bind to gp41 and gp120 protein, with the conjugation to gp41 protein stronger.

### (IV) The Action of JHR on CXCR4 Receptor by T Lymphotropic Lymphocytes and on CCR5 Co-Receptor by Macrophage-Tropic Lymphocytes

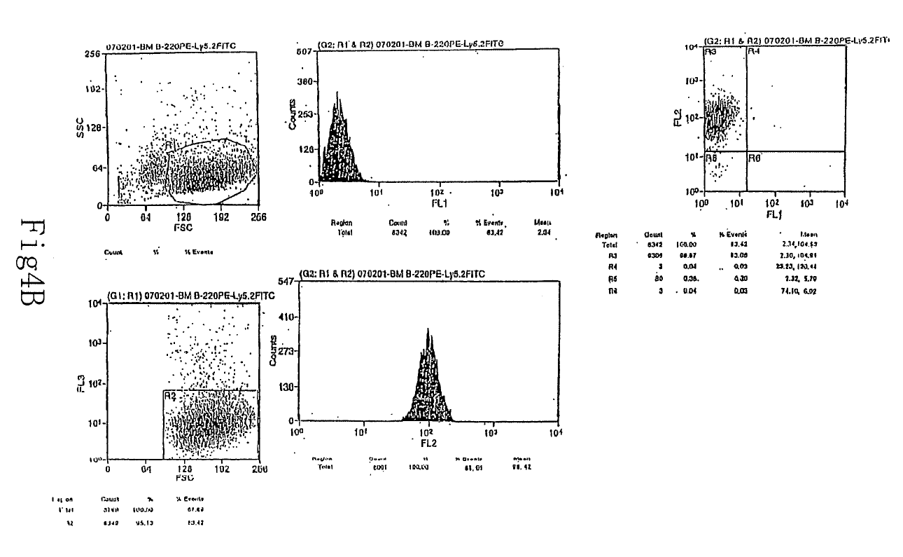

The test was carried out by using the same method as MAGI test above. (The result is shown in FIG. 5), the explanation to the result is listed in the following Table 5:

TABLE 5
The study on the co-receptor and mechanism of entry thereof (MAGI test)
T-cell lymphotropic virus strain (NL4). Hela-CD4 cell
Macrophage-tropic virus strain (Yu2). 293 T cell

| Drug | Virus | Cell strain | Results | | |
|---|---|---|---|---|---|
| Drug | NL4 | Hela-CD4 | Inhibition | No inhibition | Inhibition |
| Drug | Yu2 | 293T cell | No inhibition | Inhibition | Inhibition |
| Expla- | | | Acting on | Acting on | Acting on |
| nation | | | CXCR4 | CCR5 | CD4 or |
| | | | co-receptor | co-receptor | other |
| | | | | | mechanism |

Other mechanism: acting on, other than receptor, cell membrane, coat of virus, releasing of RNA, etc.

### (V) Action of JHR on CD4 Receptor

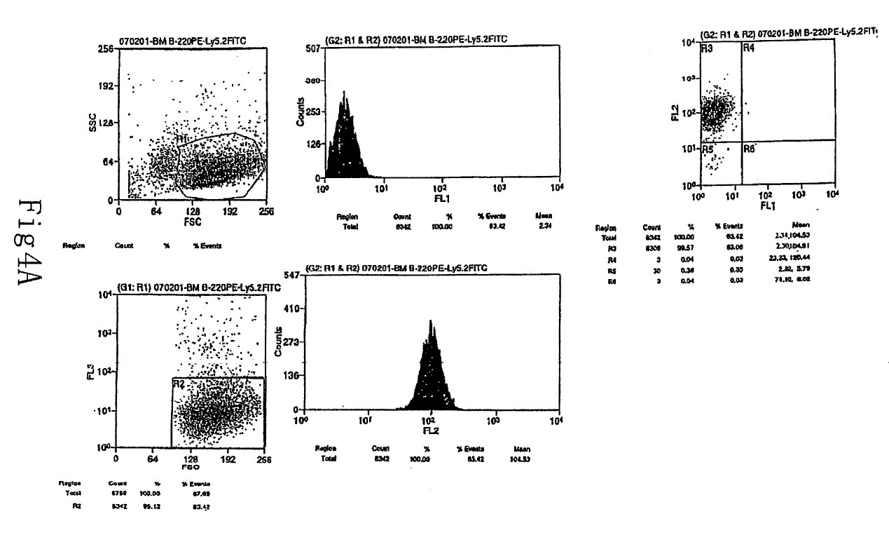

The method of Flow Cytometry was used for measurement. Method was given as follows: SupT1 cell with drug was co-incubated at 37° C. for 2 hours and washed with PBS+2% FCS. In a 4° C. ice bath, CD₄PE was added and sit for 30 minutes. After further washing and centrifuging, CD₄ monoclonal Ab was added to, followed by incubating in ice bath for 30 minutes; again after washed and centrifuged and sit in ice bath. The cells were suspended in 50 μl secondary Ab anti-mice-FITC for 20 minutes, followed by washing once and were suspended in 300-500 ul PBS/2% CS+PI. FACS testing was performed. The testing results were shown in FIGS. 4A, B.

Results showed that JHR had no effects upon CD4 receptor.

### EXAMPLE 3

### Study on Combination Medication

Object: It is to observe if there is any synergistic effect between JHR and AZT.

Method: For the experiment, the MAGI test method was adopted (the same as above).

(A) Single-agent medication: five doses of AZT were used in amount of from 1 μM to 3.9 nM, and were designated AZT1-5, five doses of JHR were designated in amount of 400 ug/ml to 1.56 ug/ml, and IC₅₀ were obtained respectively.

### (B) Combined Application

Half dosage of AZT was combined with half dosage of JHR-1 as one sample. AZT1 was combined respectively with any of JHR1-JHR5; Each of AZT2, AZT3, AZT4, AZT5 was respectively combined with any of ZN1-ZN5. So a total of 25 concentration combinations could be used. Each concentration was set in duplicate wells (2 wells). Additional group was taken as cell and virus controls.

(C) Comparing each drug combination with the virus group to have the inhibition rate. The inhibition rate of each drug combination was compared with the sole AZT IC50 to obtain the difference of functions each other.

Result: see table 6

TABLE 6

| | | |
|---|---|---|
| Drug | ED50 | AZT/A + J |

| | | | |
|---|---|---|---|
| JHR | 21.5 | μg/ml | |
| AZT | 46 | nM | |
| AZT-JHR1 | 5.8 | nM | 7.93 |
| AZT-JHR2 | 10 | nM | 4.60 |
| AZT-JHR3 | 16.7 | nM | 2.75 |
| AZT-JHR3 | 33.5 | nM | 1.37 |
| AZT-JHR-5 | 40.6 | nM | 1.13 |

As shown in Table 6, the IC50 of AZT alone is 46 nM/ml, after combining with the first dose of JHR (the composition is the same with that in example 1), IC50 of AZT is only 5.8 nM/ml, that is, ⅛ dose will obtain the same effect, showing that there is synergistic effect between JHR and AZT.

### EXAMPLE 4

### Effects on the HIV Strains Resistant to Drugs

HIV-1 was the strain resistant to protease inhibitor with virulence at 5.7×10⁴ IU/ml. The Hela-CD4 cell was adopted in MAGI test in order to observe the effects of JHR and see if there was any cross-reactivity.

Results indicated that dose of JHR was 0.4 mg/ml and the inhibition rates of virus for 5 μl or 8 μl were both as high as 100%. These demonstrated that JHR could effect upon the HIV strains resistant to protease inhibitors. The results refer to Table 5.

TABLE 5
Inhibition rate of JHR to HIV strains
resistant to protease inhibitors

| Virus load | | Drug | Inhibition rate % |
|---|---|---|---|
| PRIV | 5 μl | JHR0.4 mg/ml | 100 |
| | 8 μl | JHR0.4 mg/ml | 100 |

Note:
PRIV is the viral strain resistant to protease inhibitors.

The result shows that JHR is well effective in inhibiting HIV-1 strains resistant to protease inhibitors.

### EXAMPLE 5

### Toxicity Experiment

### I. Acute Toxicity Experiment

Result: Acute toxicity experiment indicated that no toxicity was observed for doses over 20 g/kg in intragastric dosing for rats.

### II. Sub-Acute Toxicity Experiment

Result: Sub-acute toxicity experiment indicated that after continuous intragastric dosing for 6 months, rats grew normally in the large, medium and small dose groups, ALT, BUN, RBC, WBC with DC were all normal and no abnormality was observed on pathological slides for organs such as heart, liver, kidney, spleen, lung, pancreas, brain, testis and ovary.

### INDUSTRIAL APPLICABILITY

Ginsenosides of present invention can be used to prepare drugs for treating AIDs.

What is claimed is:

1. Use of compounds of formula I in the preparation of drugs for treating AIDs, 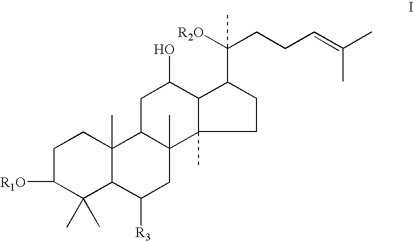
wherein, R₁ is -Glc²-Glc, R₂ is selected from the group of -Glc⁶-Glc, -Glc⁶ Ara(p), Glc⁶-Xyl and -Glc⁶-Ara(f); and R₃ is 1-1H.

2. The use according to claim 1, characterized in that R₁ is -Glc²-Glc, R₂ is -Glc⁶-Xyl, and R₃ is H.

3. The use according to claim 2, characterized in that dosages of said compounds are in amount of 0.03-0.50 mg/kg body weight.

4. The use according to any of claims 1-4, characterized in that said compounds are combined with commercialized anti-HIVs drugs.

5. The use according to claim 4, characterized in that said anti-HIVs drugs are selected from the group of AZT, DDC, DDI, Saquinavir, Ritonavir, Indinavir sulfate and Nefinavir or combination thereof.

6. The use according to claim 4, characterized in that said HIVs are those resistant to drugs repeatedly used to anti-HIV.

7. Use of composition comprising compounds of formula I as active ingredient in the preparation of drugs for treating AIDs, 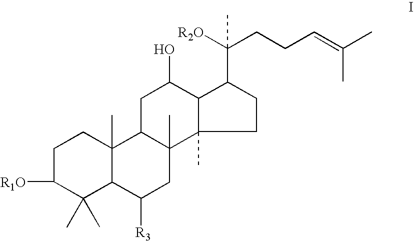
wherein R₁ is -Glc²-Glc, R₂ is selected from the group of Glc⁶-Glc, -Glc⁶-Ara(p), -Glc⁶-Xyl and -Glc⁶-Ara(f); and R₃ is H.

8. The use according to claim 7, characterized in that the composition is combined with commercialized anti-HIV drugs.

9. The use according to claim 8, characterized in that said anti-HIVs drugs are selected from the group of AZT, DDC, DPI, Saquinavir, Ritonavir, Indinavir sulfate and Nefinavir or combination thereof.

10. The use according to claim 9, characterized in that said HIVs are those resistant to drugs repeatedly used to anti-HIV.

11. Use of root, stem or leaf extracts and/or dry powder of plants selected from the group of Panax ginseng C. A. Mey, Radix panacis quinquefolii, Gymostemma pentaphyllum (thumb) makino, Panax notoginseng (burk) F. H. Chen, Panax japonicus C. A. Meyer and Panax japonicus var: major (burk.) Wu et Feng in the preparation of drugs for treating AIDs, characterized in that the extracts comprise compounds of formula I 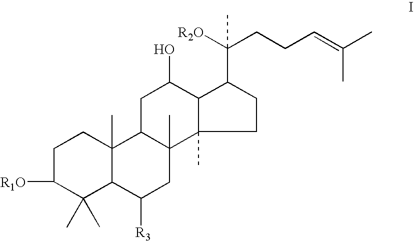
wherein, R₁ is -Glc²-Glc, R₂ is selected from the group of -Glc⁶-Glc, -Glc⁶-Ara(p), -Glc⁶-Xyl and -Glc⁶-Ara(f); and R₃ is H.

12. The use according to claim 11, characterized in that said extracts are combined with commercialized anti-HIVs drugs.

13. The use according to claim 11, characterized in that said anti-HIVs drugs are selected from the group of AZT, DDC, DPI, Saquinavir, Ritonavir, Indinavir sulfate and Nefinavir or combination thereof.

14. The use according to claim 13, characterized in that said HIVs are those resistant to drugs repeatedly used to anti-HIV.
